(19) United States
(12) Patent Application Publication
(10) Pub. No.: US 2021/0338817 A1
(43) Pub. Date: Nov. 4, 2021

(54) HEALING COMPOSITION COMPRISING ELECTROLYZED WATER

(71) Applicant: WATERDIAM GROUP LLC, Miami, FL (US)

(72) Inventors: Anthony Ginter, Miami, FL (US); Laurent PUPUNAT, Delemont (FR)

(21) Appl. No.: 17/296,153

(22) PCT Filed: Nov. 21, 2019
(86) PCT No.: PCT/EP2019/082164

§ 371 Date: May 21, 2021

(30) Foreign Application Priority Data
Nov. 21, 2018 (FR) 1871652

(51) Int. Cl. A61K 41/00; A61K 9/70; A61K 31/155; A61K 31/472; A61K 38/07; A61K 31/05; A61K 31/07; A61K 33/30; A61K 33/34; A61K 31/197; A61K 36/886; A61K 36/38; A61K 36/28; A61K 36/736; A61K 36/185; A61K 31/728; A61P 17/02

(52) U.S. Cl. CPC A61K 41/00; A61P 17/02; A61K 31/155; A61K 31/472; A61K 38/07; A61K 31/05; A61K 31/07; A61K 33/30; A61K 33/34; A61K 31/197; A61K 36/886; A61K 36/38; A61K 36/28; A61K 36/736; A61K 36/185; A61K 31/728; A61K 9/7007

(57) ABSTRACT

The present invention relates to a composition comprising electrolyzed water for use as a healing medicament. It also relates to the use of such a composition for the treatment of skin conditions.

The invention relates to the treatment of wounds and more particularly to a healing composition comprising electrolysed water obtained from a water that has undergone electrolysis in the presence of boron-doped diamond electrodes.

The healing treatment nowadays takes different forms depending on the severity of the wound to be treated.

For minor injuries that bleed, a simple bandage is often used.

There are many different types on the market, with different properties.

However, without being restrictive, mention may be made of the application WO2017027386, the object of which is a wound dressing comprising a support and a hydrophobic elastomeric matrix coating said support, said matrix comprising an elastomer of the SEPS, SEBS or SEEPS type, consisting of a combination of polystyrene blocks and polyolefin blocks, said elastomer having a molecular weight less than or equal to 290,000 Dalton measured by gel permeation chromatography, the total proportion of elastomer being strictly less than 3.0% of the total weight of said elastomeric matrix.

In the case of more serious or disabling diseases such as haemophilia, a simple wound dressing is not sufficient and requires the use of chemical or biological compounds which are often difficult to extract or expensive in order to treat the patients. Without being exhaustive, mention may be made of the application WO2012117203, which relates to a pharmaceutical composition comprising a modified factor Xa (GDXa), said modified GDXa being non-thrombogenic, able to bind to the TFPI but not able to bind to phospholipids, for the prevention or the treatment of a hemorrhagic stroke in a patient suffering from haemophilia A or B with or without an inhibitor.

Today, a more insidious disease than haemophilia is making its way into the body and causing terrible damage to patients, affecting the ability of the body to heal effectively over the long term. This 21st century disease is the diabetes of type II, which affects the patients not only in their daily lives by forcing them to inject insulin, but also in their daily struggle against wounds or cuts that have difficulty healing and sometimes lead to gangrene and are life-threatening. In the application WO2001091696, the object of the invention is the use of biguanide derivatives of general formula (I) in which the groups R1 and R2 represent, independently of each other, a hydrogen atom, a C1-C7 alkyl group, a cycloalkyl group, a heterocycle, a C2-C7 alkenyl group, an aryl group, an aralkyl group, an aryloxylalkyl group or a heteroaryl group or R1 and R2 taken together represent a C2-C7 alkylene which may contain one or more heteroatoms and the group R3 represents a primary, secondary or tertiary amine or pharmaceutically acceptable salts thereof for the manufacture of a drug having a healing effect, said drug being in a topical pharmaceutical form.

Although there is a wide therapeutic arsenal to treat wounds and heal them effectively, the use of chemicals or biological factors is never without risk, as resistance to a composition can develop over the long term. Thus, there is a real need for new compositions or compounds that effectively heal wounds without systematically resorting to complex chemical compounds or biological factors that can sometimes, but not always, induce rejection or side effects that limit or affect the healing process.

The purpose of the invention is therefore to remedy the aforementioned drawbacks and to meet the aforementioned needs by providing a composition comprising electrolysed water for use as a healing drug.

In one embodiment according to the invention the electrolysed water is obtained in the presence of at least one boron-doped diamond electrode attached to a substrate.

The concentration of boron in the diamond electrode is between 200 ppm (3×10¹⁹ B atoms/cm³) and 2,000 ppm (3.52×10²⁰ B atoms/cm³), in particular between 200 ppm (3×10¹⁹ B atoms/cm³) and 1,500 ppm (2×10²⁰ B atoms/cm³).

The substrate for fixing said diamond electrode is selected from silicon or niobium, tantalum or tungsten or a mixture thereof, preferably silicon or niobium and more preferably silicon.

According to one aspect of the invention, the electrolysed water is obtained by implementing a three-step method which starts with the use of a running or spring water optionally supplemented with sodium chloride (NaCl) at a concentration of 0.5 to 2 g/L followed by an electrolysis of said water by an electrolysis module comprising at least one boron-doped diamond electrode attached on silicon substrate in which the boron concentration is between 200 ppm (3×10¹⁹ B atoms/cm³) and 2,000 ppm (3.52×10²⁰ B atoms/cm³), in particular between 200 ppm (3×10¹⁹ B atoms/cm³) and 1,500 ppm (2×10²⁰ B atoms/cm³), said module subjecting the water to an amount of current (current density) during the electrolysis process of between 15 and 500 mAh/L of water, more preferably 40 to 250 mAh/L of water, even more preferably 50 to 200 mAh/L, the duration of the electrolysis being between 15 and 30 minutes.

Furthermore, the water used in the composition according to the invention is a natural or purified water without the need for an addition of at least one additive.

In another embodiment of the invention the composition according to the invention may also comprise a natural or synthetic healing agent.

The natural or synthetic healing agent is always present at a lower concentration than in an equivalent composition comprising conventional or distilled non-electrolysed water.

The concentration of natural or synthetic healing agent is 25-75% lower than that of an equivalent composition comprising conventional or distilled non-electrolysed water.

Preferably the natural or synthetic healing agent is selected from metformin, copper or its derivatives, papaverine, bendazole, extract of calendula, aloe vera, healing essential oils, Acetyl-Ser-Asp-Lys-Pro AcSDKP tetrapeptide, zinc or its derivatives, provitamin B5, sucralfate, resveratrol, lanolin, vitamin A, allantoin, hyaluronic acid, tocopherol or its derivatives, garden marigold, oil of sweet almond or jojoba, calophyll or St. John's wort or a mixture thereof.

The composition according to the invention also comprises one or more natural or synthetic emulsifiers and/or excipients selected from petrolatum, glycerine, paraffin, cetearyl glucose, beeswax or rice wax, soya lecithin, sugar esters, glyceryl stearate, derivatives of olive oil or a mixture thereof.

In one embodiment, the composition according to the invention comprises from 60 to 95 parts by weight of electrolysed water, from 0 to 10 parts by weight of synthetic or natural healing agent and from 1 to 10 parts by weight of an excipient and/or emulsifier.

In another embodiment, the composition according to the invention comprises from 60 to 95 parts by weight of an excipient and/or emulsifiers, from 0 to 10 parts by weight of synthetic or natural healing agent and from 10 to 30 parts by weight of electrolysed water.

The composition according to the invention may also be in the form of cream, gel or emulsion.

The object of the invention is also the use of a composition according to the invention for the treatment of conditions of the skin generating wounds or healing defects, such as bedsores, erysipelas, open wounds, varicose ulcers.

The electrolysed water forming the basis of the composition of the invention can advantageously be obtained by a method using an electrolysis module comprising at least one boron-doped diamond electrode attached on a silicon substrate in which the boron concentration is between 200 ppm (3×10¹⁹ B atoms/cm³) and 2,000 ppm (3.52×10²⁰ B atoms/cm³), in particular between 200 ppm (3×10¹⁹ B atoms/cm³) and 1,500 ppm (2×10²⁰ B atoms/cm³), said module subjecting the water to an amount of current during the electrolysis process of between 15 and 500 mAh/L of water, more preferably 40 to 250 mAh/L of water, even more preferably 50 to 200 mAh/L.

The duration of the electrolysis can be more or less long, generally less than or equal to 60 minutes, in particular between 15 and 60 minutes, in particular between 15 and 30 minutes. Typically, a duration of between 5 and 30 minutes is a reasonable time. The electrolysis can be carried out in cycles, between 4 and 12 water treatment cycles per 24 hours.

The invention also has as its final object an adhesive-type application, a wound dressing, a patch or even a mask for the skin comprising or being obtained from a composition according to the invention.

The composition of the invention will in particular enable to produce a mask which can be applied to the skin or the face, said mask being obtained from a powder such as a clay, an exfoliant, a powdered plant extract or a clay containing active charcoal and to which electrolysed water, obtained according to the method described above, has been added.

The attached drawings illustrate the invention:

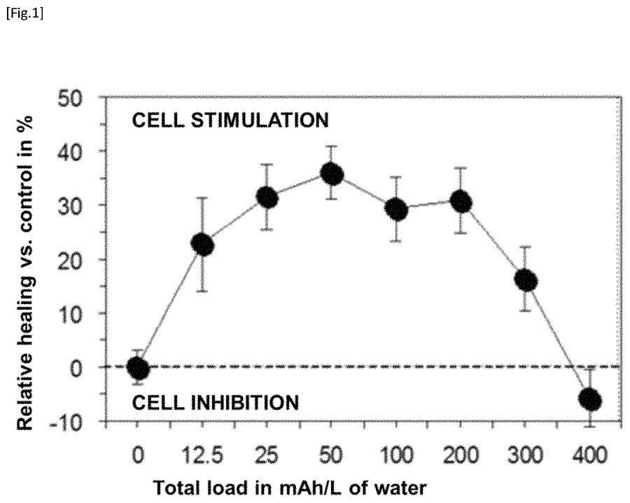

FIG. 1 represents the action of a composition according to the invention comprising electrolysed water obtained by electrolysis of water by boron-doped diamond electrodes A (1,200 ppm boron) on a silicon substrate on the healing process of a wound.

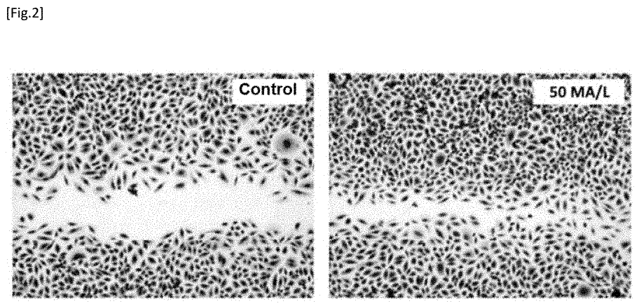

FIG. 2 represents a comparative test between a treatment with a conventional water (control) and a water electrolysed in a composition according to the invention on the healing process.

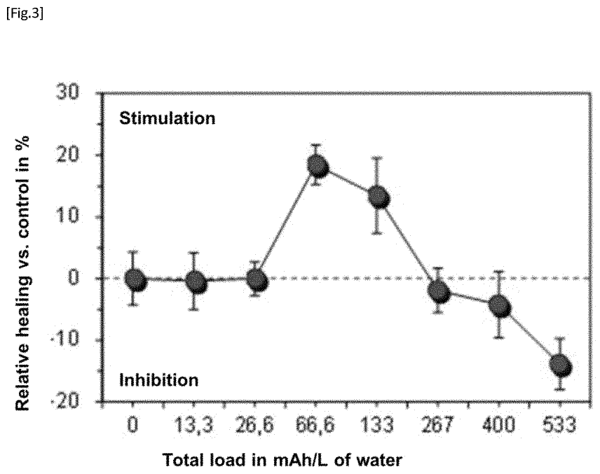

FIG. 3 represents the action of electrolysed water obtained by electrolysis of water by boron-doped diamond electrodes B (2,500 ppm boron) on a silicon substrate on the healing process of a wound.

The present invention will be described in more detail and with the aid of one or more examples which are in no way limiting to the invention.

The wound healing is a complex mechanism, involving many proteins and cellular mechanisms of reconstruction of the wound. The process can be broken down into three phases; an early vascular and inflammatory phase, also known as debridement phase or exudative phase for the debridement of a wound, a second phase known as the granulation phase, which corresponds to the proliferative phase with development of the granulation tissue, the tissue enabling the loss of substance to be filled by a new tissue thanks to the neo-angiogenesis and the cell proliferation. The granulation phase continues until the epithelialisation. This is followed by a longer phase known as scar remodelling.

Several solutions exist on the market that can be used depending on the severity of the wounds to be treated. But a simple scratch is not treated in the same way as a varicose ulcer. It is therefore useful to have new treatments available compared to those conventionally used, mainly based on the use of natural or synthetic chemical compounds or wound dressings.

The advantage of the present invention is that the use of drugs is reduced or even eliminated for superficial or more complex healing and is simple to implement due to the simplicity of the composition. By its very nature, said composition can also be “reactivated”. Such reactivation is understood to mean the ability to repeatedly apply an electrolysis treatment to the composition, which allows the water, useful product present in the composition, to be regenerated. In addition, a significant advantage is that the cost of the composition due to its raw material is lower than that of the conventional compositions. The easy accessibility of the main product comprised in the composition according to the invention is also one of these advantages.

The composition according to the invention useful in the treatment of the wound healing or associated disorders is mainly composed by an electrolysed water obtained by using a portable or fixed membraneless electrolysis station comprising at least one boron-doped diamond electrode fixed on a substrate which can be made of silicon, niobium, tantalum, tungsten or a mixture thereof.

According to the invention, the use of silicon or niobium substrates is recommended, the silicon being the preferred substrate.

Without being bound by the theory, the boron-doped diamond (called BDD) electrodes on a silicon substrate allow high electrolysis potentials to be achieved, higher than the platinum electrodes conventionally used for the water electrolysis. Thus, these BDD electrodes allow to generate structural modifications that will give to the water a therapeutic potential and more particularly on the healing processes.

In a preferred embodiment of the invention the concentration of boron present in the electrodes is between 200 ppm (3×10¹⁹ B atoms/cm³) and 1,500 ppm (2×10²⁰ B atoms/cm3) constituting an optimum in order to obtain a quality electrolysed water.

A lower boron level do not result in a quality electrolysed water and a higher boron level leads to a reduction in the performance of the electrodes and a degradation in the lifetime of the electrodes.

The electrolysed water based on the inventive composition is obtained by a method using an electrolysis module comprising at least one boron-doped diamond electrode fixed on a silicon substrate as previously mentioned, said module subjecting the water to an amount of current applied to the water during the electrolysis process of between 15 and 500 mAh/L of water, more preferably 40 to 250 mAh/L of water, still more preferably 50 to 200 mAh/L.

The duration of electrolysis can be of varying time, between 5 and 30 minutes. The electrolysis can be carried out in cycles, between 4 and 12 cycles of water treatment per 24 hours can be envisaged.

Without being bound by the theory, the water obtained by the method described above is not characterizable as such and it is this water obtained by the method that has the healing properties according to the invention. For example, the use of a water obtained by the implementation of the method described above, obtained using not a boron-doped diamond electrode but a conventional platinum electrode, does not enable to obtain a composition according to the invention and its advantageous effects.

In the composition according to the invention, the electrolysed water can be used just before making said composition and, advantageously, can be electrolysed again at any time, thus maintaining the healing potential of said composition for a very long time and somehow regenerating said composition by a relatively simple method.

The water used may be a commercially available, purified or thermal water known for its therapeutic properties or virtues. The electrolysed water don't need to be a distilled water. Furthermore, the invention does not require the addition of at least one additive such as those used in electrolysed water preparation methods used to clean the swimming pools or the spas, i.e. high concentrations of chlorine or active oxygen.

Furthermore, one of the advantages of using water electrolysed by a BDD type electrode in a composition according to the invention is that in the presence of a known natural or synthetic healing agent, the presence of the latter enables to use less natural or synthetic healing agent than if a composition containing it alone were used in the presence of a non-electrolysed or distilled natural or running water.

Thus, in compositions according to the invention which may comprise such agents, the natural or synthetic healing agent will always be present at a lower concentration than in an equivalent composition comprising a conventional or distilled non-electrolysed water. This has the advantage of reducing the dependence or ineffectiveness of a product in the long term. For some diseases, the wound healing is a recurring problem, and the prolonged or regular exposure may cause the body to become accustomed to or resistant to these agents.

The concentration of natural or synthetic healing agent in the composition according to the invention will be 25-75% lower than that of an equivalent commercially available composition not comprising electrolysed water but a conventional or distilled water.

As mentioned above, the composition according to the invention may also comprise a natural or synthetic healing agent. It should be noted that the latter is not to be understood as an additive in the framework of the present invention, as the additives as mentioned above are to be understood as additives of an organic or inorganic type in the form of salts used to purify the water.

The natural or synthetic healing agent is understood in the framework of the present invention as products which have been recognized in patents or scientific literature, known to the person skilled in the art in the field of healing products as products likely to be used as healing agents or in related diseases such as skin conditions generating wounds or healing defects, such as bedsores, erysipelas, open wounds, varicose ulcers.

Without being exhaustive, the natural or synthetic healing agent may be selected from metformin, copper or its derivatives, papaverine, bendazole, extract of calendula, aloe vera, healing essential oils, AcSDKP, zinc or its derivatives, provitamin B5, sucralfate, resveratrol, lanolin, vitamin A, allantoin, hyaluronic acid, tocopherol or its derivatives, garden marigold, oil of sweet almond or jojoba, calophyll or St. John's wort or a mixture thereof.

The composition according to the invention may also comprise natural or synthetic excipients and/or emulsifiers, in accordance with the galenic principles known to the person skilled in the art aimed at putting the composition into cream form. Without being exhaustive, these may be selected from petrolatum, glycerine, paraffin, cetearyl glucose, beeswax or rice wax, soya lecithin, sugar esters, glyceryl stearate, oil derivatives, more particularly olive oil derivatives, or a mixture thereof.

Depending on the type of cream to be obtained and on its desired penetrating power, a higher or lower percentage of electrolysed water can be added to the cream in relation to the emulsifiers or vice versa. In the case of a water-in-oil emulsion, also known as W/O, the quantity of oil is greater than the quantity of water. The emulsion thus obtained is very nourishing, moisturising and protective because it creates a lipidic film on the skin. It is ideally used for the dry skin or the night creams.

In the case of an emulsion called oil-in-water, also known as O/W, the quantity of water is greater than the quantity of oil. This type of emulsion is nourishing and moisturising. It is ideally used to make day creams and body milks, and can also be incorporated into patches or bandage-type adhesives.

Another potential galenic form for the composition according to the invention can be an aqueous gel also called hydrogel. A hydrogel is a gel in which the swelling agent is water. The matrix of a hydrogel is generally a network of polymers which are insoluble in water, but are capable of substantially swelling in the presence of a large amount of water or aqueous solutions.

Thus, several formulations or shaping of the composition according to the invention may be possible depending on the needs of the patient to be treated and the extent of the healing process to be implemented. It may be in the form of a cream, gel or emulsion or even in the form of a bandage-type adhesive or a patch for the skin.

Depending on the type of emulsion sought or composition sought, the composition according to the invention in the case of a so-called O/W composition comprises 60 to 95 parts by weight of electrolysed water, 0 to 10 parts by weight of synthetic or natural healing agent and 1 to 10 parts by weight of an excipient and/or emulsifiers.

In the case of a so-called W/O composition, said composition comprises from 60 to 95 parts by weight of an excipient and/or emulsifiers, from 0 to 10 parts by weight of synthetic or natural healing agent and from 10 to 30 parts by weight of electrolysed water.

The various possible galenic formulations of the composition according to the invention are made for use as healing drug or the treatment of skin conditions generating wounds or healing defects, such as bedsores, erysipelas, open wounds or varicose ulcers.

### EXAMPLES

### Example 1: Study of the Effect of the Electrode Boron Concentration on the Healing Efficiency of a Wound

The rate of healing of fibroblasts as a function of the boron content of the electrodes used to electrolyse the culture water was studied.

The boron-doped electrodes (hereafter referred to as BDD/Si electrodes) A and B implemented in this experiment have the following characteristics:
BDD/Si electrodes: boron-doped diamond film on silicon substrate:
Substrate: single crystal silicon (100), resistivity 100 mohm·cm
BDD film: polycrystalline, thickness ^{˜}2-3 μm, doping 1,200 ppm boron (Electrode A) or 2,500 ppm boron (Electrode B),

The electrodes A and B were manufactured using the same HF-CVD (Hot Filament Chemical Vapor Deposition) diamond film growth protocol. They are identical in every respect and differ only in their respective boron content.

a) The Protocol for Implementing the Electrodes to the Culture Water is as Follows:

A 2.5 L tank contains city water at 15° C. This water is pumped at a fixed rate of 200 L/h through an electrolysis module and then returned to the tank in a closed circuit. The electrolysis module uses 2 electrodes spaced 1 mm apart and with an active surface area of 70 cm². The electrolysis current is 2 A for working periods t=0-1-2-5-10-20-30-40 min so as to achieve electrolysis loads of 0 to 533 mAh/L. The water of the tank is kept at a constant temperature of 20° C. during the test.

The electrolysed water is sampled at the outlet of electrolysis module and then immediately sterile filtered (0.2 μm porous membrane filters) and added to the fibroblast culture medium at a 1:4 dilution (=25% concentration).

b) The Culture Protocol is as Follows:

Fibroblasts: L-929 (mouse fibroblasts; ACC 173; DSMZ); internal passage P52-53; recommended according to EN ISO 10993-5: 2009). The cells are incubated and cultured in mass in a 37° C. incubator with a controlled closed environment containing 5% of CO2 and 95% of air. The culture medium is the RPMI 1640 with 10% physiological bovine serum, 100 Unit/mL penicillin and 100 μg/mL streptomycin.

c) The Protocol for Studying the Cell Regeneration/Healing is as Follows:

Use of silicone culture inserts (ibidi GmbH, München). When this insert is placed in a culture medium, it forms 2 culture tanks separated by a 500 μm thick wall. The cells are grown in both tanks and the silicone insert is removed. This results in two perfectly defined culture patches, precisely 500 μm apart.

For the experiments, L-929 type cells were obtained from 80-90% by mass of suspension cultures at a density of 500,000 cells/ml. 100 μL of suspension are introduced into each culture insert tank. The cells are grown for 24 hours to obtain homogeneous populations in each of the two tanks of each insert. Then, the insert is gently removed, leaving a 500 μm free space separating the two culture media. The electrolysed water is injected at a ratio of 500 μL to 1,500 μL of fresh culture medium (1:4 dilution). The culture media are grown again for 24 hours. Then, layers of cells were fixed by a methanol treatment for 2 min and stained by means of a Coomassie-Giemsa solution according to Romanowsky.

The separation space is photographed via a 27″ screen in order to observe at 5 points along the space, the closing speed of the two media, up to the junction (FIG. 2).

d) Results

The results with the water electrolysed with the BDD electrode A (1,200 ppm boron doping B) show a significant acceleration of the healing rate compared to the sterile water without electrolysis, up to more than 30% from 25 mAh/L to 200 mAh/L and then a decrease (FIG. 1).

With the BDD electrode B (approx. 2,500 ppm B), an acceleration of the healing is observed, but much lower than the one obtained with the electrodes A (FIG. 3).

### Example 2=Boron Content of the Electrolysed Water

The protocol for electrolysis of tap water is as follows:

The water used is a city water.

The electrolysis protocol is as follows:

The city water is pumped at a rate of 90 L/h through an electrolysis module equipped with 2 BDD electrodes A as defined in example 1 (1,200 ppm B) spaced 1 mm apart, having 12.5 cm² of active surface.

The applied current is 2.4 A. The water sample is collected directly from the outlet of the electrolysis module (open loop, wastewater operation).

The results are reported in the tables below:

Sample 1: raw city water

Samples 2 and 3: electrolysed water with electrodes A (1,200 ppm boron)

| Sample | Boron | Si |
|---|---|---|
| 1 raw city | 31.5 | 4.7 |
| 2 mini cell low rate 2.4A | 34 | 4.54 |
| 3 mini cell high rate 2.2A | 34.2 | 4.54 |
| | μg/L | mg/L |
| release | 2.60 | μg B/L |
| average rate | 90 | L/h |
| average current | 2.3 | A |
| | 101.74 | μg B/Ah |

The electrolysis of water with electrodes A causes a small but noticeable increase in the boron concentration in the electrolysed water compared to the raw water: approximately +101.74 microgram boron/Ah of applied electrical charge.

The boron measurements in water are performed by Inductively Coupled Plasma Mass Spectrometry (ICP-MS).

1. A composition comprising electrolysed water, wherein the electrolysed water is obtained by electrolysis using at least one boron-doped diamond electrode attached to a substrate, wherein the concentration of boron is between 200 (3×10¹⁹ B atoms/cm³) and 2,000 ppm (3.52×10²⁰ B atoms/cm³), in particular between 200 ppm (3×10¹⁹ B atoms/cm³) and 1,500 ppm (2×10²⁰ B atoms/cm³).

2. The composition according to claim 1, wherein the water is a natural or purified water without addition of at least one additive.

3. The composition according to claim 1, further comprising a natural or synthetic healing agent.

4. The composition according to claim 3, wherein the natural or synthetic healing agent selected from metformin, copper or its derivatives, papaverine, bendazole, extract of calendula, aloe vera, essential healing oils, Acetyl-Ser-Asp-Lys-Pro (AcSDKP) tetrapeptide, zinc or its derivatives, provitamin B5, sucralfate, resveratrol, lanolin, vitamin A, allantoin, hyaluronic acid, tocopherol or its derivatives, garden marigold, oil of sweet almond, jojoba or St. John's wort or a mixture thereof.

5. The composition according to claim 1, wherein there are also present natural or synthetic emulsifiers and/or excipients selected from petrolatum, glycerine, paraffin, cetearyl glucose, beeswax or rice wax, soya lecithin, sugar esters, glyceryl stearate, derivatives of olive oil or a mixture thereof.

6. The composition according to claim 1, wherein the composition comprises from 60 to 95 parts by weight of electrolysed water, from 0 to 10 parts by weight of synthetic or natural healing agent and from 1 to 10 parts by weight of an excipient and/or emulsifiers.

7. The composition according to claim 1, wherein, the composition comprises from 60 to 95 parts by weight of an excipient and/or emulsifiers, from 0 to 10 parts by weight of synthetic or natural healing agent and from 10 to 30 parts by weight of electrolysed water.

8. The composition according to claim 1, wherein the composition is used as a healing drug in the treatment of skin conditions generating wounds or healing defects, such as bedsores, erysipelas, open wounds, varicose ulcers.

9. A skin application mask comprising a composition comprising electrolysed water, wherein the electrolysed water is obtained by electrolysis using at least one boron-doped diamond electrode attached to a substrate, wherein the concentration of boron is between 200 (3×10¹⁹ B atoms/cm³) and 2,000 ppm (3.52×10²⁰ B atoms/cm³), in particular between 200 ppm (3×10¹⁹ B atoms/cm³) and 1,500 ppm (2×10²⁰ B atoms/cm³).
